(19) United States
(12) Patent Application Publication
(10) Pub. No.: US 2020/0016376 A1
(43) Pub. Date: Jan. 16, 2020

(54) CATHETER SYSTEM FOR IMPROVED FIRST STICK SUCCESS

(71) Applicant: Becton, Dickinson and Company, Franklin Lakes, NJ (US)

(72) Inventors: Mohankumar Natesan, Bukit Batok (SG); Ong Xue Guang John, Ghim Moh (SG)

(21) Appl. No.: 16/032,640

(22) Filed: Jul. 11, 2018

(51) Int. Cl. A61M 25/06

(52) U.S. Cl. CPC A61M 25/0612; A61M 25/0606; A61M 25/0693

(57) ABSTRACT

A pediatric catheter system may include a catheter adapter having a distal end, a proximal end, and a lumen extending therebetween. The system may include a catheter tube extending distally from the catheter adapter. The catheter tubing may be constructed of thermoplastic polyurethane for improved vein insertion success. The system may include a needle hub removably coupled to a proximal end of the catheter adapter and an introducer needle extending through the catheter tube. A proximal end of the introducer needle may be secured within the needle hub. The introducer needle may include a notch, which may also facilitate successful vein insertion. In response to insertion of the catheter tube into a vein of a patient, blood may flow into the introducer needle and out of the notch into a space disposed between an outer surface of the introducer needle and an inner surface of the catheter tube.

### BACKGROUND

Infusion therapy, a common healthcare procedure, may be facilitated by a vascular access device. Hospitalized, home care, and other patients receive fluids, pharmaceuticals, and blood products via a vascular access device inserted into the vascular system. Blood withdrawal is another common healthcare procedure that may be facilitated by a vascular access device.

A vascular access device may access a peripheral or central vasculature of a patient. A vascular access device may be indwelling for short term (days), moderate term (weeks), or long term (months to years). A vascular access device may be used for continuous infusion therapy or for intermittent therapy.

A common type vascular access device is an over-the-needle peripheral intravenous catheter (PIVC). As its name implies, the “over-the-needle” PIVC may be mounted over an introducer needle having a sharp distal tip. The sharp distal tip may be used to pierce skin and the vasculature of the patient. Insertion of the PIVC into the vasculature may follow the piercing of the vasculature by the needle. The needle and the PIVC are generally inserted at a shallow angle through the skin into the vasculature of the patient with a bevel of the needle facing away from the skin of the patient.

Particularly in pediatric patients, including neonates, where veins may be smaller and more difficult to access, multiple needle sticks may occur in an attempt to position the PIVC in a vein. Thus, in some instances, more vulnerable patients, including children, may receive more needle sticks, which may result in pain and other complications. Once the catheter is placed within the vein, the needle may be removed, leaving the catheter in place for future blood withdrawal or fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a catheter system configured to facilitate catheter insertion success, as well as related devices and methods. In further detail, in some embodiments, the catheter system may reduce complications that may be associated with insertion of a catheter tube into a vein of a patient and/or improve first needle stick success. In some embodiments, the catheter system may be particularly useful for pediatric patients, including neonates, to reduce a risk that these more vulnerable patients experience more than a single needle stick.

In some embodiments, a catheter system may include a catheter adapter having a distal end, a proximal end, and a lumen extending therebetween. In some embodiments, the system may include a catheter tube extending distally from the catheter adapter. In some embodiments, the catheter tubing may be constructed of thermoplastic polyurethane for improved vein insertion success. In some embodiments, the system may include a needle hub removably coupled to a proximal end of the catheter adapter and an introducer needle extending through the catheter tube. In some embodiments, a proximal end of the introducer needle may be secured within the needle hub. In some embodiments, the needle hub may include a thumb grip, which may extend outwardly from a distal end of the needle hub.

In some embodiments, the introducer needle may include a notch, which may also facilitate successful vein insertion. In some embodiments, in response to insertion of the catheter tube into a vein of a patient, blood flashback may flow into a distal tip of the introducer needle and out of the notch into a space disposed between an outer surface of the introducer needle and an inner surface of the catheter tube. In some embodiments, the catheter tube may be transparent, which may facilitate visualization of the blood flashback within the catheter tube. The notched needle, which may provide instant blood flashback, may be particularly important in pediatric patients with small veins and where there is a delay in blood flashback reaching a flow control plug, for example. The notched needle providing instant blood flashback may also be important in catheter systems with a non-transparent catheter adapter for gauge identification purposes, because instant flashback may be provided distal to the catheter adapter.

In some embodiments, the catheter system may include a pediatric catheter system. In some embodiments, the catheter tube may be 24 gauge or 26 gauge. In some embodiments, a length of the catheter tube may be approximately 19 mm. In some embodiments, the catheter tube may include a peripheral intravenous catheter.

In some embodiments, the catheter system may include the flow control plug, which may be coupled to a proximal end of the needle hub. In some embodiments, the flow control plug may be vented. In some embodiments, in response to insertion of the catheter tube into the vein of the patient, blood flashback may flow into the flow control plug. In some embodiments, the flow control plug may be transparent, which may facilitate visualization of blood flashback within the flow control plug. Thus, in some embodiments, the catheter system may allow visualization of blood flashback both within the catheter and within the flow control plug, providing multiple places within the catheter system to visualize the blood to determine the catheter is placed properly within the vein.

In some embodiments, the catheter adapter may be non-transparent. In some embodiments, the catheter adapter may include one or more wings, which may extend outwardly from the catheter adapter. In some embodiments, the wings may be configured to fold upwardly. In some embodiments, the catheter system may include a compartment, which may be removably coupled to the catheter adapter. In some embodiments, the compartment may fit over the catheter adapter and secure the wings in an upward position. In some embodiments, the compartment may include a finger grip, which may extend outwardly from a distal end of the compartment.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a top view of an example catheter assembly, according to some embodiments;

FIG. 2A is a side view of an example catheter system, according to some embodiments;

FIG. 2B is a top view of the catheter system of FIG. 2A, according to some embodiments;

FIG. 2C is a bottom view of the catheter system of FIG. 2A, according to some embodiments;

FIG. 2D is a front view of the catheter system of FIG. 2A, according to some embodiments;

FIG. 2E is a rear view of the catheter system of FIG. 2A, according to some embodiments;

FIG. 2F is a cross-sectional view of the catheter system of FIG. 2A, according to some embodiments;

FIG. 3 is a front view of an example compartment of the catheter system of FIG. 2A, according to some embodiments;

FIG. 4 is a cross-sectional view along the line 4-4 of FIG. 3, the compartment mounted on an example catheter adapter, according to some embodiments;

FIG. 5 is an exploded view of the catheter system of FIG. 2A, according to some embodiments; and

FIG. 6 is a cross-sectional view of an example needle in an insertion position within an example catheter, according to some embodiments.

### DESCRIPTION OF EMBODIMENTS

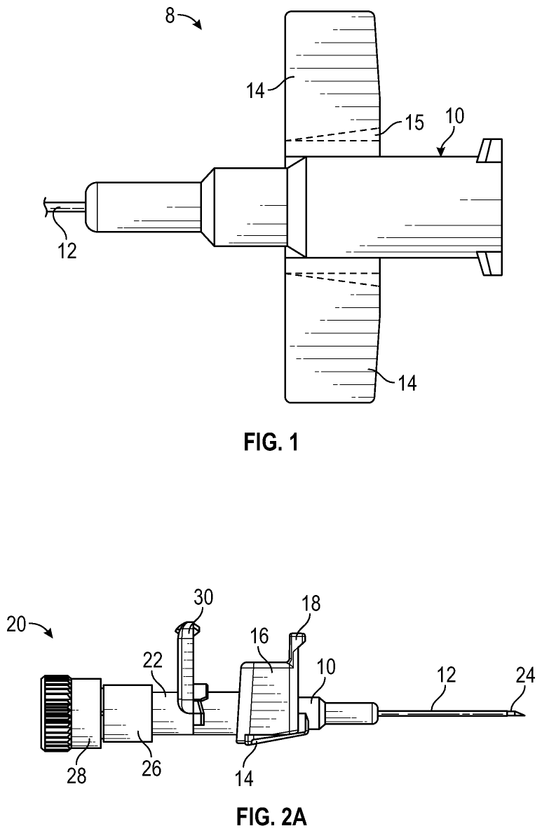

Referring now to FIG. 1, an example catheter assembly 8 is illustrated, according to some embodiments. In some embodiments, the catheter assembly 8 may be similar to the BD Neoflon™ IV Cannula System or another suitable catheter system.

In some embodiments, the catheter assembly 8 may include a catheter adapter 10 and a catheter tube 12. In some embodiments, the catheter tube 12 may extend distally from the catheter adapter 10. In some embodiments, the catheter tube 12 may include a small gauge catheter sized for a pediatric patient, which may include, for example, a neonatal patient.

For example, the catheter tube 12 may have a gauge size of 24. In these and other embodiments, the catheter tube 12 may include an external diameter approximately equal to 0.7 mm and/or a length approximately equal to 19 mm. In some embodiments, a water flow rate through the catheter tube 12 may be approximately 20 ml/min. As another example, the catheter tube 12 may have a gauge size of 26. In these and other embodiments, the catheter tube 12 may include an external diameter approximately equal to 0.6 mm and/or a length approximately equal to 19 mm. In some embodiments, a water flow rate through the catheter tube 12 may be approximately 13 ml/min.

In some embodiments, the catheter tube 12 may be constructed of thermoplastic polyurethane (“TPU”) or another suitable material. In some embodiments, the catheter tube 12 may be constructed solely or partially of TPU. In some embodiments, the TPU or other suitable material may facilitate a smooth insertion of the catheter tube 12 into the vein of the patient. In some embodiments, the TPU or other suitable material may provide less resistance as a user inserts the catheter tube 12 into the vein than a standard catheter tube. A standard catheter tube may be constructed of polytetrafluoroethylene (“PTFE”) or a similar material, which may be difficult to insert into the vein of the patient, providing resistance which may lead to collapse of the standard catheter tube. In some embodiments, the catheter tube 12 constructed of TPU or other suitable material may ease insertion into the vein of the patient and help avoid collapse of the catheter tube 12 during insertion. In some embodiments, the catheter tube 12 constructed of TPU may reduce a risk of phlebitis and extend an indwelling period of the catheter tube 12.

In some embodiments, the catheter adapter 10 may include one or more wings 14, which may extend outwardly from the catheter adapter 10. In some embodiments, the wings 14 may extend from a lower portion of the catheter adapter 10. In some embodiments, the catheter adapter 10 may be constructed of a relatively flexible plastic. In some embodiments, the wings 14 may include a scoring 15 to facilitate the wings 14 folding to an upward and/or downward position. In some embodiments, a color of the catheter adapter 10 may indicate to the user a gauge of the catheter tube 12. For example, a yellow catheter adapter 10 may indicate a 24 gauge catheter tube 12 or a purple catheter adapter 10 may indicate a 26 gauge catheter tube 12. Using a catheter adapter color to indicate a gauge size to a user may be common in the art.

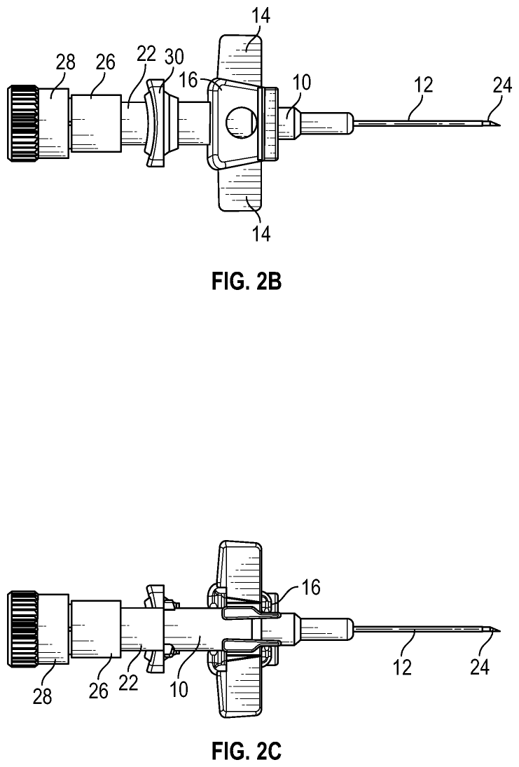

Referring now to FIGS. 2A-2F, a catheter system 20 is illustrated, according to some embodiments. In some embodiments, the catheter system 20 may include one or more of the following: the catheter adapter 10, the catheter tube 12, the compartment 16, a needle hub 22, a needle 24, a flow control plug 26, and an end cap 28. In some embodiments, the colored catheter adapter 10 may be important to indicate to the user that the catheter tube 12 is sized for pediatric patients, including neonatal patients or older children, such that a larger gauge designed for an adult is not used.

In some embodiments, a compartment 16 may be removably coupled to the catheter adapter 10. In some embodiments, the compartment 16 may include a finger grip 18 configured to aid in insertion and/or withdrawal of the catheter assembly 8 from a patient. In some embodiments, the compartment 16 may be generally square. In some embodiments, the compartment 16 may be useful for the user, such as a clinician, to grip during insertion of the catheter tubing 12 into the vein of the patient. In some embodiments, the compartment 16 may provide a larger surface area to grip as compared to the catheter adapter 10 itself.

In some embodiments, a proximal end of the needle 24 may be secured within the needle hub 22. In some embodiments, the proximal end of the needle 24 may be press-fit within the needle hub 22. In some embodiments, the needle 24 may include an introducer needle having a sharp distal tip to facilitate insertion of the catheter tube 12 into a vein of the patient. In some embodiments, the needle hub 22 may include a thumb grip 30, which may be configured to aid in removal of the needle hub 22 and needle 24 from the catheter assembly 8, after the catheter tube 12 is inserted into the vein of the patient. In some embodiments, the needle 24 may be constructed of stainless steel or another suitable material.

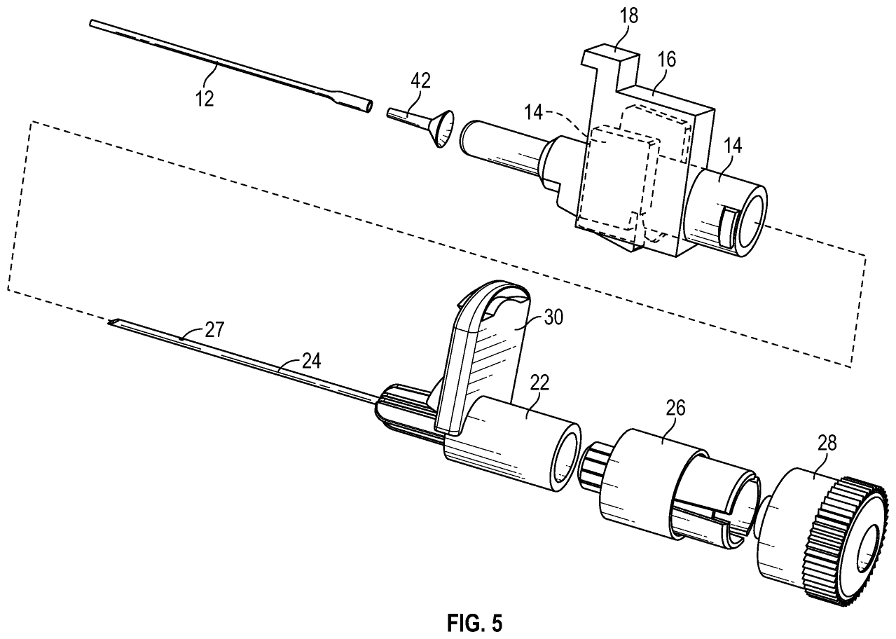

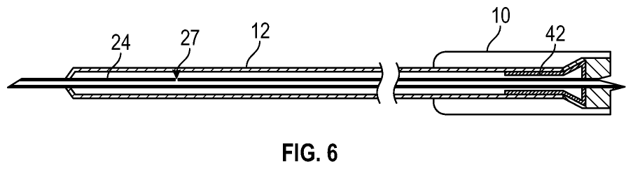

In some embodiments, the needle 24 may include a notch 27 (illustrated, for example, in FIGS. 5-6) towards a distal end of the needle 24, which may provide flashback indicating that the catheter tube 12 has been properly placed within the vein of the patient. In further detail, in some embodiments, in response to the needle 24 being inserted into the vein of the patient, flashback of blood may flow through the sharp distal tip of the needle 24 and out of the notch 27 into a portion of the catheter system 20. For example, the blood flashback may flow through the distal tip and out of the notch 27 into a space between an exterior surface of the needle 24 and an interior surface of the catheter tube 12 (this may be referred to as “primary flashback”). In some embodiments, blood flashback may flow into other locations of the catheter system 20, such as, for example, an extension tube (not illustrated) coupled to the catheter adapter 10. The primary blood flashback may confirm that the needle 24 is located within the vein of the patient.

In some embodiments, the flow control plug 26 may include a reservoir 29, which may be fluidly connected to a proximal end of the needle 24. Thus, in some embodiments, blood flashback may flow through the needle 24 and exit the proximal end of the needle (or a notch in the proximal end of the needle 24) to enter the needle hub 22 and flow control 26. Additionally or alternatively, in some embodiments, blood flashback that exits the notch 27 may fill the reservoir.

In some embodiments, the flow control plug 26 may be transparent. Thus, in some embodiments, the blood flashback disposed within the flow control plug 26 (“secondary flashback”) may be visible to the user. In some embodiments, the catheter system 20 may include the blood flashback visible within the catheter as well as the flashback visible within the flow control plug 26, which may enhance visibility of the flashback to the user. Generally, flashback in the BD Neoflon™ IV Cannula System may be visible in the particular flow control plug of the BD Neoflon™ IV Cannula System, but the BD Neoflon™ IV Cannula System may not include the primary flashback feature. Flashback within the flow control plug 26 may take several seconds to see, but the notch 27 may provide instant flashback or “instaflash” to support quick vein confirmation.

In some embodiments, the flow control plug 26 may be configured to vent air, such as, for example, with a semi-permeable membrane, porous membrane, and/or micro grooves disposed within the flow control plug 26, but may contain blood. In some embodiments, an end cap 28 may be coupled to the proximal end of the flow control plug 26. In some embodiments, the end cap 28 may prevent venting of the flow control plug 26 when the end cap 28 is secured to the flow control plug 26. In some embodiments, the end cap 28 and/or proximal end of the flow control plug 26 may include a luer adapter, such as a slip or thread male or female luer adapter, or another suitable connector, which may facilitate coupling of the end cap 28 and the flow control plug 26.

In some embodiments, a lumen may extend from a distal end of the catheter adapter 10 to a proximal end of the catheter adapter 10. In some embodiments, a proximal end of the catheter adapter 10 may include a luer adapter, such as a slip or thread male or female luer adapter, or another suitable connector. In some embodiments, a distal end of the needle hub 22 may include a connector configured to couple with the connector of the proximal end of the catheter adapter 10. For example, the distal end of the needle hub 22 may include a luer adapter, such as a slip or thread male or female luer adapter, or another suitable connector.

The catheter system 20 of FIGS. 2A-2E is illustrated in a position corresponding to after insertion of the catheter tube 12 into the vein of the patient but prior to removal of the needle 24, according to some embodiments. In some embodiments, in this position, the wings 14 may be disposed in a downward position for securement to skin of the patient via adhesive, tape, or another suitable mechanism. In some embodiments, when the catheter system 20 is in an insertion position prior to inserting the catheter system 20 into the patient, the wings 14 may be disposed in an upward position within the compartment 16, as will be explained in further detail.

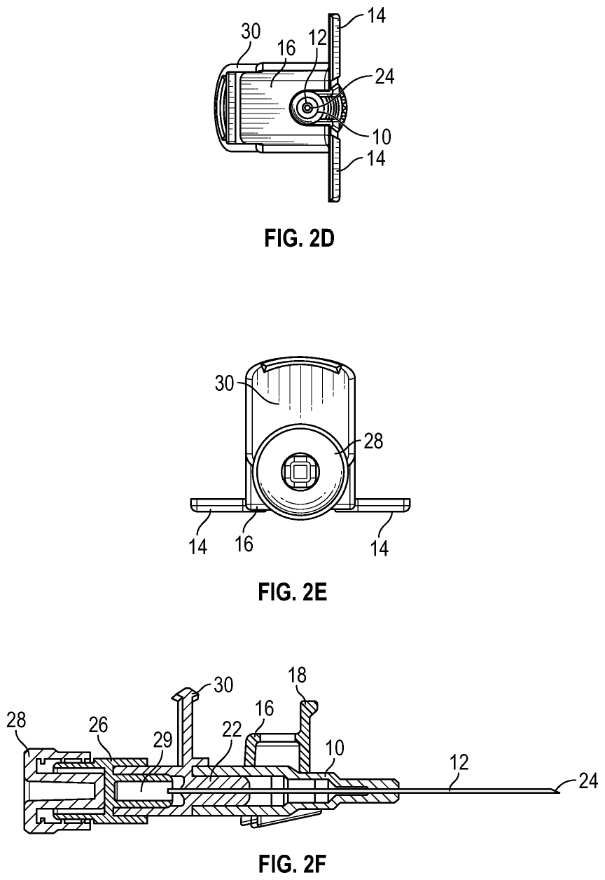

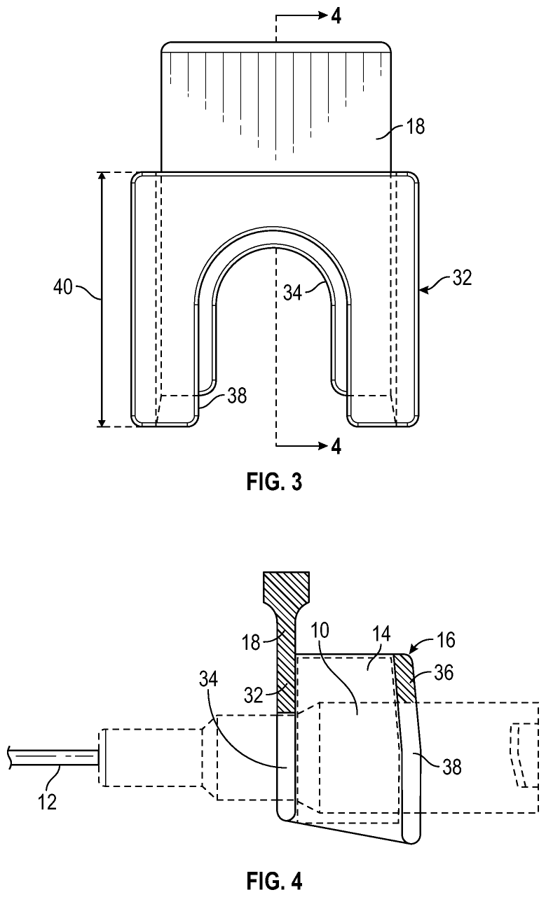

Referring now to FIG. 3, in some embodiments, the compartment 16 may include a distal wall 32, which may include a first arched gate 34, and proximal wall 36 (illustrated, for example, in FIG. 4), which may include a second arched gate 38. In some embodiments, the first arched gate 34 and the second arched gate 38 may each include a width that is slightly larger than a diameter of the catheter adapter 10, such that the compartment 16 may be pushed with a sliding fit over the catheter adapter 10. In some embodiments, the gates 34, 38 may embrace the catheter adapter 10 ahead of and/or behind the wings 14. In some embodiments, a height 40 of the compartment 16 may be at least a same dimension as a length of wings 14.

Referring now to FIG. 4, in some embodiments, the wings 14 may be folded upwardly, and then the compartment 16, having an opening in at least a bottom portion, may be pushed with the sliding fit over the catheter adapter 10, securing the wings 14 within the compartment 16. In some embodiments, after the normal manipulations, i.e. vein puncture, positive insertion of the catheter tube 12 into the vein, and separation of the needle hub 22 from the catheter adapter 10, have been carried out by means of the finger grip 18 and/or the thumb grip 30, the compartment 16 may be removed from the catheter adapter 10, and the wings 14 may be unfolded and then fixed to skin of the patient.

Referring now to FIG. 5, a wedge 42 is illustrated, according to some embodiments. In some embodiments, the wedge 42 may secure the catheter tube 12 within the catheter adapter 10. In some embodiments, the wedge 42 may be constructed of metal or another suitable material.

Referring now to FIG. 6, in some embodiments, the needle 24 may include the notch 27, which may facilitate blood flashback. FIG. 6 illustrates the wedge 42 securing the catheter tube 12 within the catheter adapter 10, according to some embodiments.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A pediatric catheter system, comprising:
a catheter adapter having a distal end, a proximal end, and a lumen extending therebetween;
a catheter tube extending distally from the catheter adapter, wherein the catheter tube is constructed of thermoplastic polyurethane;
a needle hub removably coupled to a proximal end of the catheter adapter; and
an introducer needle extending through the catheter tube, wherein a proximal end of the introducer needle is secured within the needle hub.

2. The pediatric catheter system of claim 1, wherein the catheter tube is 24 gauge or 26 gauge.

3. The pediatric catheter system of claim 1, wherein a length of the catheter tube is approximately 19 mm.

4. The pediatric catheter system of claim 1, wherein the introducer needle comprises a notch disposed towards a distal end of the introducer needle, wherein in response to insertion of the catheter tube into a vein of a patient, blood flows into a distal end of the introducer needle and out of the notch into a space disposed between an outer surface of the introducer needle and an inner surface of the catheter tube, wherein the catheter tube is transparent.

5. The pediatric catheter system of claim 4, further comprising a flow control plug coupled to a proximal end of the needle hub, wherein the flow control plug is vented, wherein in response to insertion of the catheter tube into the vein of the patient, blood flows into the flow control plug, wherein the flow control plug is transparent.

6. The pediatric catheter system of claim 5, wherein the catheter adapter is non-transparent.

7. The pediatric catheter system of claim 1, wherein the needle hub comprises a thumb grip extending outwardly from a distal end of the needle hub.

8. The pediatric catheter system of claim 1, wherein the catheter adapter further comprises a plurality of wings, further comprising a compartment, wherein the plurality of wings extend outwardly from the catheter adapter, wherein the plurality of wings are configured to fold upwardly, wherein the compartment fits over the catheter adapter and secures the plurality of wings in an upward position.

9. The pediatric catheter system of claim 7, wherein the compartment comprises a finger grip extending outwardly from a distal end of the compartment.

10. A pediatric catheter system, comprising:
a catheter adapter having a distal end, a proximal end, and a lumen extending therebetween;
a catheter tube extending distally from the catheter adapter;
a needle hub removably coupled to a proximal end of the catheter adapter; and
an introducer needle extending through the catheter tube, wherein a proximal end of the introducer needle is secured within the needle hub, wherein the introducer needle comprises a notch disposed towards a distal end of the introducer needle, wherein in response to insertion of the catheter tube into a vein of a patient, blood flows into a distal end of the introducer needle and out of the notch into a space disposed between an outer surface of the introducer needle and an inner surface of the catheter tube.

11. The pediatric catheter system of claim 10, further comprising a flow control plug that is vented, wherein in response to insertion of the catheter tube into the vein of the patient, blood flows into the flow control plug, wherein the flow control plug is transparent.

12. The pediatric catheter system of claim 11, wherein the catheter adapter is non-transparent, the flow control plug is transparent, and the catheter tubing is transparent.

13. The pediatric catheter system of claim 12, wherein the catheter tube is constructed of thermoplastic polyurethane, wherein the catheter tube is 24 gauge or 26 gauge.

14. The pediatric catheter system of claim 10, wherein the needle hub comprises a thumb grip extending outwardly from a distal end of the needle hub.

15. The pediatric catheter system of claim 10, wherein the catheter adapter further comprises a plurality of wings, further comprising a compartment, wherein the plurality of wings extend outwardly from the catheter adapter, wherein the plurality of wings are configured to fold upwardly, wherein the compartment fits over the catheter adapter and secures the plurality of wings in an upward position.

16. The pediatric catheter system of claim 10, wherein the compartment comprises a finger grip extending outwardly from a distal end of the compartment.

17. A pediatric catheter system, comprising:
a catheter adapter having a distal end, a proximal end, and a lumen extending therebetween;
a catheter tube extending distally from the catheter adapter, wherein the catheter tube is constructed of thermoplastic polyurethane, wherein the catheter tube is 24 gauge or 26 gauge;
a needle hub removably coupled to a proximal end of the catheter adapter; and
an introducer needle extending through the catheter tube, wherein a proximal end of the introducer needle is secured within the needle hub, wherein the introducer needle comprises a notch disposed towards a distal end of the introducer needle, wherein in response to insertion of the catheter tube into a vein of a patient, blood flows into a distal end of the introducer needle and out of the notch into a space disposed between an outer surface of the introducer needle and an inner surface of the catheter tube; and
a flow control plug that is vented, wherein in response to insertion of the catheter tube into the vein of the patient, blood flows into the flow control plug,
wherein the catheter adapter is non-transparent, the flow control plug is transparent, and the catheter tubing is transparent.

18. The pediatric catheter system of claim 17, wherein the needle hub comprises a thumb grip extending outwardly from a distal end of the needle hub.

19. The pediatric catheter system of claim 17, further comprising a plurality of wings and a compartment, wherein the plurality of wings extend outwardly from the catheter adapter, wherein the plurality of wings are configured to fold upwardly, wherein the compartment fits over the catheter adapter and secures the plurality of wings in an upward position.

20. The pediatric catheter system of claim 19, wherein the compartment comprises a finger grip extending outwardly from a distal end of the compartment.
